(19) United States
(12) Patent Application Publication
(10) Pub. No.: US 2026/0325610 A1
(43) Pub. Date: Oct. 22, 2026

(54) GLUCAGON-LIKE-PEPTIDE-2 (GLP-2) ANALOGUES AND THEIR MEDICAL USES FOR THE TREATMENT OF SHORT BOWEL SYNDROME (SBS)

(71) Applicant: Zealand Pharma A/S, Søborg (DK)

(72) Inventors: Mikkel Askjær AGERSNAP, Søborg (DK); Thor Schütt Svane NIELSEN, Søborg (DK)

(21) Appl. No.: 19/116,558

(22) PCT Filed: Sep. 29, 2023
(86) PCT No.: PCT/EP2023/077065

§ 371 Date: Mar. 28, 2025

Related U.S. Application Data
(60) Provisional application No. 63/377,868, filed on Sep. 30, 2022.

(51) Int. Cl. A61K 38/26; A61P 1/00

(52) U.S. Cl. CPC A61K 38/26; A61P 1/00

(57) ABSTRACT

The present invention relates to glucagon-like-peptide-2 (GLP-2) analogues, and in particular to ZP1848 (glepaglutide), and their medical uses for the treatment of short bowel syndrome (SBS), and in particular to the treatments using glepaglutide that result in an early or significant reduction in the parenteral support (PS) required by patients undergoing GLP-2 therapy, even allowing patients to be weaned off the need for PS. In combination with a subpopulation of patients becoming SBS non-PS, the patients further reported an improvement in their Quality-of-Life (QoL).

### FIELD OF THE INVENTION

The present invention relates to glucagon-like-peptide-2 (GLP-2) analogues and their medical uses for the treatment of short bowel syndrome (SBS), and in particular to the treatments using glepaglutide that result in an early or significant reduction in the parenteral support (PS) required by patients undergoing GLP-2 therapy, even allowing patients to be weaned off the need for PS.

### BACKGROUND OF THE INVENTION

Human GLP-2 is a 33-amino-acid peptide with the following sequence: Hy-His-Ala-Asp-Gly-Ser-Phe-Ser-Asp-Glu-Met-Asn-Thr-Ile-Leu-Asp-Asn-Leu-Ala-Ala-Arg-Asp-Phe-Ile--Asn-Trp-Leu-Ile-Gln-Thr-Lys-Ile-Thr-Asp-OH (SEQ ID NO: 2). It is derived from specific post-translational processing of proglucagon in the enteroendocrine L cells of the intestine and in specific regions of the brainstem. GLP-2 binds to a single G-protein-coupled receptor belonging to the class II glucagon secretin family.

GLP-2 has been reported to induce significant growth of the small intestinal mucosal epithelium via the stimulation of stem cell proliferation in the crypts, and by inhibition of apoptosis in the villi (Drucker et al., 1996, Proc. Natl. Acad. Sci. USA 93: 7911-7916). GLP-2 also has growth effects on the colon. Furthermore, GLP-2 inhibits gastric emptying and gastric acid secretion (Wojdemann et al., 1999, J. Clin. Endocrinol. Metab. 84: 2513-2517), enhances intestinal barrier function (Benjamin et al., 2000, Gut 47: 112-119), stimulates intestinal hexose transport via the upregulation of glucose transporters (Cheeseman, 1997, Am. J. Physiol. R1965-71), and increases intestinal blood flow (Guan et al., 2003, Gastroenterology, 125: 136-147). For a review of GLP-2 and its properties, see Burrin et al., 2001, The Journal of Nutrition, 131(3), March 2001, 709-712.

It has been recognised in the art that glucagon-like peptide-2 receptor analogues have therapeutic potential for the treatment of intestinal diseases. However, the native hGLP-2, a 33 amino acid gastrointestinal peptide, is not a useful in a clinical setting due to its very short half-life in humans of around 7 minutes for full length GLP-2 [1-33] and 27 minutes for truncated GLP-2 [3-33]. In large part, the short half-life is due to degradation by the enzyme dipeptidylpeptidase IV (DPP-IV). Accordingly, there have been attempts in the art to develop GLP-2 receptor agonists with better pharmacokinetic characteristics, in particular to improve the half-life of GLP-2 molecules. By way of example, GLP-2 analogues with substitutions have been suggested such as e.g., GLP-2 analogues containing Gly substitution at position 2 ([hGly2] GLP-2, teduglutide) which increases the half-life from seven minutes (native GLP-2) to about two hours. Teduglutide is approved for treatment of short bowel syndrome under the names Gattex (in the US) and Revestive (in Europe).

Jeppesen et al. (Gut, 60: 902-14, 2011, “Randomised placebo-controlled trial of teduglutide in reducing parenteral nutrition and/or intravenous fluid requirements in patients with short bowel syndrome”) reports a 24-week placebo-controlled study that evaluated the safety of teduglutide and its ability to reduce parenteral support in patients with SBS with intestinal failure. The trial confirmed that teduglutide was safe and well tolerated, but the other end points of the trial, in particular regarding any possible reduction in PS failed due to statistics.

Jeppesen et al. (Gastroenterology, 143: 1473-81, 2012, “Teduglutide reduces need for parenteral support among patients with short bowel syndrome with intestinal failure”) reports a placebo-controlled STEP study in which a 24-week study of patients with SBS-IF were given subcutaneous teduglutide. In the study, a significant reduction in PS reduction was reported but no patients were completely weaned from PS at week 24.

O'Keefe et al. (Clin. Gastroenterol. Hepatol., 11: 815-23, 2013, “Safety and efficacy of teduglutide after 52 weeks of treatment in patients with short bowel intestinal failure”) reports a 28-weeks double blinded extension study in which patients were treated for 52 weeks in total, investigating the long-term safety, tolerability, and clinical efficacy of teduglutide in patients with SBS-IF during the 52-week treatment period. As this was an extension study it included only active arms and thus was not placebo controlled.

WO 2006/117565 (Zealand Pharma A/S) describes GLP-2 analogues which comprise one of more substitutions as compared to [hGly2] GLP-2 and which improved biological activity in vivo and/or improved chemical stability, e.g. as assessed in in vitro stability assays. Among the molecules disclosed in WO 2006/117565 is ZP1848 (glepaglutide) which has been designed to be stable in liquid formulations. Dosage regimes for GLP-2 analogues including ZP1848 and its metabolites are described in WO 2018/229252, which also shows that these compounds are effective to increase longitudinal growth of the intestines.

Use of GLP-2 analogues, including ZP1848, to treat conditions associated with bile acid synthesis, liver bile acid content, or intestinal bile acid content is described in WO 2020/020904.

Ready-to use formulations of ZP1848 are described in WO 2020/065064.

### SUMMARY OF THE INVENTION

Broadly, the present invention is based on the surprising findings arising from the EASE SBS 1 and EASE SBS 2 trials, which are multi-center, placebo controlled, randomized, parallel-group, double-blind phase 3 clinical trials (NCT:03690206 and NCT:03905707) that investigated at the safety and effectiveness of treatment for short bowel syndrome (SBS) using glepaglutide. These placebo-controlled studies found that ZP1848 treatment in patients with short bowel syndrome receiving parenteral support (PS), in particular those receiving PS for 3 or more days per week, allowed a subpopulation of patients to be weaned entirely from the parenteral support after only a few weeks of treatment. These findings are significant as they the first results in a placebo-controlled trial to show that weaning off the PS is possible in a subpopulation of patients. This is significant given the clinical challenges in devising and adjusting PS regimes for patients, the side effects and complications associated with it and because it significantly impacts the quality of life of patients receiving PS.

The results of this EASE SBS 1 trial also included the surprising finding that a secondary end point of the trial using Patient Global Impression of Change (PGIC), a PRO (patient-report outcome) tool, where patients rate their change in overall status since start of the trial on a 7-point Likert scale (see https://www.fda.gov/media/116277/download and https://www.fda.gov/media/116281/download), led to improvement and significant differences relative to placebo for both glepaglutide when administered twice weekly (TW, p=0.0020) and once weekly (OW, p<0.0001), for example when PGIC was assessed at week 24. This meant that of the subpopulation of patients weaned off PS, 8/9 of them reported a PGIC status that was “much improved” or “very much improved”.

Accordingly, in a first aspect, the present invention provides a glucagon-like peptide 2 (GLP-2) analogue for use in a method for the treatment of a human patient suffering from short bowel syndrome (SBS) and receiving parenteral support (PS), wherein the GLP-2 analogue is represented by the formula:

H-HGEGTFSSELATILDALAARDFIAWLIATKITDKKKKKK-NH₂,(1848) (SEQ ID NO: 1)
or a pharmaceutically acceptable salt thereof, the method comprising:
   (a) providing a human patient suffering from short bowel syndrome and receiving parenteral support (PS);
   (b) for a period of time administering by subcutaneous injection once or twice weekly 10 mg of a GLP-2 analogue into the patient, while the patient continues to receive the PS; and
   (c) following step (b), weaning the patient off the PS.

In a further aspect, the present invention provides a method of treating short bowel syndrome in a patient in need thereof said method including: (a) providing a human patient suffering from short bowel syndrome and receiving parenteral support (PS); (b) for a period of time administering by subcutaneous injection once or twice weekly 10 mg of a GLP-2 analogue into the patient, wherein the GLP-2 analogue is represented by the formula:

(SEQ ID NO: X)
H-HGEGTFSSELATILDALAARDFIAWLIATKITDKKKKKK-NH₂,
(1848)

or a pharmaceutically acceptable salt thereof
while the patient continues to receive the PS; and (c) following step (b), weaning the patient off the PS.

In a further aspect, the present invention provides the use of a glucagon-like peptide 2 (GLP-2) analogue in the preparation of a medicament for the treatment of a human patient having short bowel syndrome (SBS) and receiving parenteral support (PS), wherein the GLP-2 analogue is represented by the formula:

(SEQ ID NO: 1)
H-HGEGTFSSELATILDALAARDFIAWLIATKITDKKKKKK-NH₂,
(1848)

or a pharmaceutically acceptable salt thereof, the treatment comprises:
   (a) provision of parenteral support to the human patient having short bowel syndrome;
   (b) administration for a period of time subcutaneous injection once or twice weekly 10 mg of a GLP-2 analogue into the patient, while the patient continues to receive the PS; and
   (c) following step (b), weaning the patient off the PS.

In these aspects of the present invention, the period of time in step (b) is between 20 weeks and 52 weeks (e.g., between 20 weeks and 30 weeks, between 24 weeks and 36 weeks, between 24 weeks and 48 weeks, between 28 weeks and 42 weeks, or between 30 weeks and 48 weeks). In some cases, the treatment time during which the GLP-2 analogue is administered is 20 weeks, 24 weeks, 28 weeks, 30 weeks, 36 weeks, 42 weeks, 48 weeks, or 52 weeks. Typically, a preferred duration of treatment is 24 weeks, and this represents a time point at which the benefits of weaning off PS and/or an improvement in QoL were clinically observed.

The medical uses or methods can further include: (d) following step (c) administering by subcutaneous injection once or twice weekly 10 mg of the GLP-2 analogue into the patient while the patient receives no PS.

In any of the above medical uses or methods, the patient is treated once or twice weekly. In some cases, the patient is treated for at least 1 year, or at least 2 years, or at least 3 years or for a duration of at least 1 to 3 years. In some cases, treatment with the GLP-2 analogue may be continued indefinitely.

The subpopulation of patients achieving complete weaning off PS is typically at least 5%, or at least 6% or at least 7% or at least 8% or at least 10% of the full cohort treated with the GLP-2 analogue. In preferred aspects, the patients at the start of the treatment receive PS on 2 or more days a week, 3 or more days a week, 4 or more days a week, 5 or more days a week, 6 or more days a week or daily.

The subpopulation of patients achieving complete weaning off PS may be additionally characterised by an improvement in their quality of life (QoL). As set out herein, this may be assessed using a Patient Global Impression of Change (PGIC) status, for example with responding patients reporting a much improved or very much improved status as compared to treatment with placebo. Generally, the improved PGIC status of the patient is observable at week 24 of the treatment. The subpopulation of patients achieving an improvement in QoL in addition to complete weaning off PS is preferably at least 50%, or at least 60% or at least 70% or at least 75%, at least 80%, or at least 85% of the subpopulation achieving complete weaning off PS.

In any of the medical uses or methods, step (b) may include administering by subcutaneous injection once weekly 10 mg of the GLP-2 analogue into the patient. In any of the medical uses or methods, step (b) may include administering by subcutaneous injection twice weekly 10 mg of the GLP-2 analogue into the patient.

In some cases, in any of the medical uses or methods, the patient has undergone an end-jejunostomy or ileostomy.

In some cases, in any of the medical uses or methods, the patient has undergone a jejuno-colic anastomosis.

In some cases, in any of the medical uses or methods, the patient has undergone a jejuno-ileo-colic anastomosis.

In some cases, step (b) includes the patient receiving PS at an ESPEN guideline level of any one of A1, B1, C1, D1, A2, B2, C2, D2, A3, B3, C3, D3, A4, B4, C4, or D4 (see Table 1).

In the present context, the term “parenteral support” or “PS” includes the provision of nutrients and/or fluids to the patient receiving GLP-2 therapy as a means of providing the patient with the nutrients and/or fluids that they require but are unable to absorb fully due to their condition.

In the present context, the term “weaning off” refers to a patient who had been receiving PS and GLP-2 therapy for improving their gastrointestinal function such that all PS is withdrawn (i.e., PS ceases to be a part of their treatment regimen).

In the present context, the terms achieving “oral or enteral autonomy” refers to SBS patients who had been receiving PS and GLP-2 therapy to improve their gastrointestinal function to the extent that all PS is withdrawn (i.e., PS ceases to be a part of their treatment regimen).

The terms “patient” and “patient” are used interchangeably in this specification. It will be understood that the patient (or patient) is a mammal, and typically a human.

ZP1848 is also effective in increasing intestinal mass and longitudinal intestinal growth, particularly in the small intestine.

The individual doses may be for administration via a dosing regime as described elsewhere in this specification.

WO 2018/229252 describes that ZP1848 has an unexpectedly long half-life which may enable alternative regimes such as once or twice weekly administration, especially when delivered by subcutaneous injection. Without wishing to be bound by theory, it is believed that the half life of ZP1848 may be due to the combination of the formation of a subcutaneous depot and the formation of metabolites which are slowly released from the subcutaneous depot, and which are also agonistic on the GLP-2 receptor. The subcutaneous depot may be formed on administration through a reaction between the lysine tail of ZP1848 and hyaluronic acid in the subcutaneous compartment.

Thus, the dosing regime may comprise a plurality or course of doses separated in time by 2 days, 2.5 days, 3 days, 3.5 days, 4 days, 5 days, 6 days, 7 days, 8 days, 9 days, 10 days, 11 days or 12 days. In a preferred embodiment, the doses are separated in time by 3 days, 3.5 days, 4 days, 5 days, 6 days, 7 days, or 8 days. In a preferred embodiment, doses are separated in time by 3 days, 3.5 days, 4 days, or 7 days. As will be appreciated in the art, the time between doses may be varied to some extent so that each and every dose is not separated by precisely the same time. This will often be directed under the discretion of the physician. Thus, doses may be separated in time by a clinically acceptable range of times, e.g., from about 2 days to about 10 days, or from about 3 or 4 days to about 7 or 8 days.

Such doses may be appropriate for any dosage regime, but particularly a once or twice weekly dosage regime.

In a course of treatment, the doses taken by the patient may either be the same or different in accordance with the instructions from the physician.

In some cases, it may be desirable to divide a total dose into a plurality (e.g. two or three) separate doses or administrations, for example for administration at spaced apart injection sites, for example spacing the injection sites at least 5 cm apart. Such spatially separate administrations will typically be provided at substantially the same time, e.g. on the same day, within one hour of each other, or even closer in time.

Preferably, the doses of the GLP-2 analogues used in accordance with the present invention are in the range between 0.5 mg and 25 mg per patient once or twice weekly, optionally between 1 mg and 20 mg per patient once or twice weekly, optionally between 1 mg and 10 mg per patient once or twice weekly, optionally between 2 mg and 7 mg per patient once or twice weekly, optionally between 5 mg and 7 mg per patient once or twice weekly, or optionally between 2 mg and 5 mg per patient once or twice weekly. In one embodiment, the dose of the GLP-2 analogues used in accordance with the present invention is 10 mg per patient once or twice weekly. In a course of treatment, the doses taken by the patient may either be the same or different in accordance with the instructions from the physician.

Preferably, the glucagon-like peptide 2 (GLP-2) analogue are administered to patients by injection, most typically by subcutaneous injection or intramuscular injection. In some preferred embodiments, the GLP-2 analogue may be administered using an injection pen, which allow patients to self-administer the analogue. In some aspects, administration of the GLP-2 analogue causes formation of a subcutaneous depot from which the GLP-2 analogue, or metabolites thereof, are released. Without wishing to be bound by any particular explanation, the subcutaneous depot may form through the interaction of the GLP-2 analogues administered in accordance with the present invention, in particular where the analogues comprise a lysine tail, through a reaction between the analogues and with hyaluronic acid in the subcutaneous compartment.

In the present context, the term “parenteral support” or “PS” includes the provision of nutrients and/or fluids to the patient receiving GLP-2 therapy as a means of providing the patient with the nutrients and/or fluids that they require, but are unable to absorb fully due to their condition. The determination of the correct amount or volume of PS to provide to patients with SBS who are receiving GLP-2 therapy is a challenge because if PS volume is not adjusted in a timely and appropriate way, patients may experience fluid overload, are at risk of dehydration and may not achieve optimal clinical responses to the therapy. This is further complicated as the PS volume required by a patient will typically vary during the course of GLP-2 therapy depending on their response to the therapy. Typically, the assessment of the amount of PS volume required by the patient as GLP-2 therapy progresses is dependent on how long the therapy has continued and the responsiveness of individual patients to it. In view of this variation, an initial assessment of PS volume may be carried out within the first few days of GLP-2 therapy and is typically then followed by a weekly assessment during the first month, a monthly assessment over the next 1-3 months, and thereafter an assessment every 3-6 months until the treatment is concluded. This is important as patients may experience a rapid initial response to GLP-2 therapy, improving the function of the small intestine, for example even before any increase in the length of the intestine is observed. This in turn enables the PS volume to be reduced, thereby avoiding the risk of side effects, such as fluid overload. The side effects and complications of PS are set out in Bielawska and Allard (Nutrients, 9: 466-480, 2017; doi:10.3390/nu9050466) and Cuerda et al. (Clinical Nutrition, 40: 5196-5220, 2021, doi.org/10.1016/j.clnu.2021.07.002). Aside from the side effects and complications associated with PS, it known to have a significant impact on the quality of life (QoL) of patients whose treatment regimes need to include it.

Accordingly, as a patient's need for PS reduces the method or uses may include the step of (a) determining the volume of PS required by the patient at that point in the treatment, (b) comparing it to a baseline PS volume determined at the start of therapy with the GLP-2 analogue and (c) reducing the frequency or volume of the PS where the patient demonstrates improved function of the intestine, such as the small intestine. Optionally, the reduction of a frequency or volume of the parenteral support (PS) can be performed using the algorithm described in the examples. As set out in the examples, in aspects of the present invention, a subpopulation of patients achieved complete weaning off PS.

By way of illustration of the relationship between the amount of parenteral support required by patients and the degree of improvement in intestinal function, it is presently believed that a 40% increase in length and width of the small intestine would result in at least a further 10% improvement in function or absorptive capacity of the small intestine. Generally, the GLP-2 therapy according to the present invention leads to improved function or absorptive capacity of the small intestine and as shown in the examples, the present invention surprisingly found that a subpopulation of patients treated with the GLP-2 analogue could be weaned completely off the need for any parenteral support.

One of the challenges facing patients and physicians when initiating GLP-2 therapy, namely the appropriate and individualized adjustment in volume of parenteral support (PS) provided to the patient. This is important because if the PS volume is not adjusted in a timely and appropriate way, patients may experience fluid overload, risks of dehydration and may not achieve optimal clinical responses with the therapy.

For example, in a previous 24-week treatment study (see Center for Drug and Evaluation and Research, application number 203441Orig1s000, page 16) with the GLP-2 analogue teduglutide, an attempt to reduce the parenteral nutrition volume by 10% was done at the earliest at week 4, 8, 12, 16, and 20 after start of treatment, if the urine output increased by at least 10% from baseline. Many of the patients in this study suffered from fluid overload and stopped drinking (Jeppesen et al. 2011, Gut 2011; 60:902-914). In a follow-up study, a 24-week study of patients with SBS-IF who were given subcutaneous teduglutide (Jeppesen et al. 2012, Gastroenterology 2012; 143:1473-1481), the reduction of parenteral nutrition to at least 10%, but not more than 30%, was recommended every week if the urine output increases by at least 10% from baseline. However, in this study, the patients also suffered from fluid overload, in particular at the beginning of the treatment.

Embodiments of the present invention will now be described by way of example and not limitation. However, various further aspects and embodiments of the present invention will be apparent to those skilled in the art in view of the present disclosure.

Unless context dictates otherwise, the descriptions and definitions of the features set out above are not limited to any particular aspect or embodiment of the invention and apply equally to all aspects and embodiments which are described.

### BRIEF DESCRIPTION OF THE FIGURE

FIG. 1 depicts the trial design described in Example 1. This phase 3 trial was a pivotal, multicenter, placebo controlled, randomized, parallel-group, double-blind and fixed dose, designed to confirm the efficacy of glepaglutide in reducing the parenteral support (PS) volume in SBS patients and to evaluate the efficacy of glepaglutide on other efficacy endpoints as well as the safety and tolerability of glepaglutide in patients with SBS. A 3-arm treatment, parallel-group design with a 1:1:1 randomization scheme (2 active treatment groups [once and twice weekly] and placebo) was chosen to compare the dosing regimen. The clinical trial is registered at clinicaltrials.gov: NCT03690206.

### DETAILED DESCRIPTION OF THE INVENTION

Throughout the description and claims the conventional one-letter for natural amino acids are used. All amino acid residues in the compounds described are typically of the L-configuration.

### Compounds

ZP1848 is a peptide having the formula:

H-HGEGTFSSELATILDALAARDFIAWLIATKITDKKKKKK-NH₂ (SEQ ID NO: 1)

as described e.g., in WO 2006/117565. It will be understood that the N-terminal “H—” indicates a free N-terminal amine (NH₂—) group. The C-terminal “NH₂—” indicates a C-terminal amide group. The terms ZP1848 and glepaglutide may be used interchangeably.

The invention encompasses the use of pharmaceutically acceptable salts of ZP1848, as described in more detail below. Any suitable salt may be used, although acetate may be preferred.

When ZP1848 is injected into the subcutaneous (SC) compartment, two functionally active metabolites are formed, ZP2469 and ZP2711, both C-terminal truncated analogs of ZP1848. The overall PK profile of ZP1848 therefore comprises the effect of ZP1848 and its two main metabolites.

ZP2469 is a peptide having the formula:

(SEQ ID NO: 3)
H-HGEGTFSSELATILDALAARDFIAWLIATKITDK-OH

ZP2711 is a peptide having the formula:

(SEQ ID NO: 4)
H-HGEGTFSSELATILDALAARDFIAWLIATKITDKK-OH

where the N-terminal “H—” is as described above, and the C-terminal “—OH” indicates a free C-terminal carboxylic acid group.

In alternative embodiments, the present invention may administer ZP2469 and/or ZP2711 alone or combination with each other or ZP1848.

Teduglutide is a peptide having the formula:

(SEQ ID NO: 5)
H-HGDGSFSDEMNTILDNLAARDFINWLIQTKITD-OH

where the N-terminal “H—” and C-terminal “—OH” are as described above.

### Pharmaceutical Compositions and Administration

The GLP-2 analogue as used herein may be formulated as pharmaceutical compositions prepared for storage or administration, and which comprise a therapeutically effective amount of the GLP-2 analogue in a pharmaceutically acceptable carrier.

The therapeutically effective amount of the GLP-2 analogue will depend on the route of administration, the type of mammal being treated (typically human), and the physical characteristics of the specific mammal under consideration. These factors and their relationship to determining this amount are well known to skilled practitioners in the medical arts. This amount and the method of administration can be tailored to achieve optimal efficacy so as to deliver the peptide to the intestine, but will depend on such factors as weight, diet, concurrent medication and other factors, well known those skilled in the medical arts.

The GLP-2 analogue is typically present in an amount effective for prophylaxis or treatment of the relevant condition, e.g., to treat or prevent stomach and bowel-related disorders, to increase intestinal mass and/or to promote or increase longitudinal intestinal growth of the intestines in a patient.

Examples of pharmaceutically acceptable salts are described in “Remington's Pharmaceutical Sciences”, 17th edition. Ed. Alfonso R. Gennaro (Ed.), Mark Publishing Company, Easton, PA, U.S.A., 1985 and more recent editions, and in the Encyclopaedia of Pharmaceutical Technology.

Suitable salts include acid addition salts and basic salts. Examples of acid addition salts include hydrochloride salts, citrate salts, chloride salts and acetate salts. Preferably, the salt is acetate. In general, it is preferred that the salt is not a chloride salt. Examples of basic salts include salts where the cation is selected from alkali metals, such as sodium and potassium, alkaline earth metals, such as calcium, and ammonium ions ⁺N(R³)₃(R⁴), where R³ and R⁴ independently designates optionally substituted C₁₋₆-alkyl, optionally substituted C₂₋₆-alkenyl, optionally substituted aryl, or optionally substituted heteroaryl.

Acetate salts may be particularly preferred. In the present context, the term “ZP1848-acetate” refers to the ZP1848 molecule is in the form of an acetate salt. The acetate salts of ZP1848 may be represented by the formula (ZP1848), x(CH₃COOH) where x is 1.0 to 8.0, i.e., where x is 1.0, 2.0, 3.0, 4.0, 5.0, 6.0, 7.0 or 8.0. In any composition, there may be molecules with different number of acetate molecules so that x is not necessarily a whole integer. In some cases, x is from 4.0 to 8.0, x is from 6.0 to 8.0, or x is from 4.0 to 6.5. In some cases, is from x is from 4.0 to 6.0, x is from 2.0 to 7.0, x is from 3.0 to 6.0, x is from 4.0 to 6.0 or x is 4.0 to 8.0.

As is apparent to one skilled in the medical art, a “therapeutically effective amount” of the peptides or pharmaceutical compositions of the present invention may vary depending upon the age, weight and mammalian species treated, the particular compounds employed, the particular mode of administration and the desired effects and the therapeutic indication. Because these factors and their relationship to determining this amount are well known in the medical arts, the determination of therapeutically effective dosage levels, the amount necessary to achieve the desired result (e.g. of preventing and/or treating the intestine and stomach related diseases described herein, as well as other medical indications disclosed herein, or increasing intestinal mass and/or inducing or increasing longitudinal intestinal growth of the intestines in a patient) will be within the ambit of the skilled person.

In one embodiment of the invention administration of the compounds or pharmaceutical composition of the present invention is commenced at lower dosage levels, with dosage levels being increased until the desired effect of preventing/treating the relevant medical indication, such as intestine and stomach related diseases or increased longitudinal growth of the intestines, is achieved. This would define a therapeutically effective amount. Guidance on appropriate individual doses is provided elsewhere in this specification. However, the skilled person will be able to adjust these doses in the event that an alternative dosing regime is selected.

For therapeutic use, the GLP-2 analogue is formulated with a carrier that is pharmaceutically acceptable and is appropriate for delivering the peptide by the chosen route of administration. For the purpose of the present invention, peripheral parenteral routes include intravenous, intramuscular, subcutaneous, and intraperitoneal routes of administration. In one embodiment, the route of administration is the subcutaneous route or subcutaneous administration.

When administration is to be parenteral, such as intravenous, subcutaneous, or intramuscular injectable pharmaceutical compositions can be prepared in conventional forms, either as aqueous solutions or suspensions; lyophilized, solid forms suitable for reconstitution immediately before use or suspension in liquid prior to injection, or as emulsions.

Diluents for reconstitution of the lyophilized product may be a suitable buffer, e.g. selected from a histidine buffer, mesylate buffer, acetate buffer, glycine buffer, lysine buffer, TRIS buffer, Bis-Tris buffer and MOPS buffer, water, saline, dextrose, mannitol, lactose, trehalose, sucrose, lecithin, albumin, sodium glutamate, cysteine hydrochloride; or water for injection with addition of detergents, such as Tween 20, Tween 80, poloxamers e.g. pluronic F-68 or pluronic F-127, polyethylene glycol, and or with addition of preservatives such as para-, meta-, and ortho-cresol, methyl- and propylparaben, phenol, benzyl alcohol, sodium benzoate, benzoic acid, benzyl-benzoate, sorbic acid, propanoic acid, esters of p-hydroxybenzoic acid, and or with addition of an organic modifier such as ethanol, acetic acid, citric acid, lactic acid or salts thereof.

In addition, if desired, the injectable pharmaceutical compositions may contain minor amounts of non-toxic auxiliary substances, such as wetting agents, or pH buffering agents. Absorption enhancing preparations (e.g., liposomes, detergents and organic acids) may be utilized.

In one embodiment of the invention, the compounds are formulated for administration by infusion, e.g., when used as liquid nutritional supplements for patients on total parenteral nutrition therapy (for example neonatals, or patients suffering from cachexia or anorexia), or by injection, for example subcutaneously, intraperitoneal or intravenously, and are accordingly utilized as aqueous solutions in sterile and pyrogen-free form and optionally buffered to physiologically tolerable pH, e.g., a slightly acidic or physiological pH. Formulation for intramuscular administration may be based on solutions or suspensions in plant oil, e.g., canola oil, corn oil or soybean oil. These oil-based formulations may be stabilized by antioxidants e.g., BHA (butylated hydroxianisole) and BHT (butylated hydroxytoluene).

Thus, the present peptide compounds may be administered in a vehicle, such as distilled water or in saline, phosphate buffered saline, 5% dextrose solutions or oils. The solubility of the GLP-2 analogue may be enhanced, if desired, by incorporating a solubility enhancer, such as detergents and emulsifiers.

Alternative gelling agents, such as hyaluronic acid, may also be useful as depot agents.

Subcutaneous administration may be particularly preferred, e.g., by injection.

The therapeutic dosing and regimen most appropriate for patient treatment will of course vary with the disease or condition to be treated, and according to the patient parameters.

Without wishing to be bound by any particular theory, it is expected that doses, between 0.1 and 25 mg per patient, and shorter or longer duration or frequency of treatment may produce therapeutically useful results, such as a statistically significant increase particularly in small bowel mass. In some instances, the therapeutic regimen may include the administration of maintenance doses appropriate for preventing tissue regression that occurs following cessation of initial treatment. The dosage sizes and dosing regimen most appropriate for human use may be guided by the results obtained by the present invention and may be confirmed in further clinical trials.

A human dose of ZP1848 may be used in a dose of between about 0.01 mg/kg and 100 mg/kg body weight, such as between about 0.01 mg/kg and 10 mg/kg body weight, for example between 10-100 g/kg body weight. In further embodiments, a human dose (total dose) of ZP1848 may be from about such as between and including 0.1 mg and 25 mg per patient between and including 0.5 mg and 20 mg per patient, such as between and including 1 mg and 15 mg per patient, such as between and including 1 mg and 10 mg per patient once or twice weekly or as a plurality of doses as defined herein separated in time by 2, 3, 4, 5, 6, 7, 8, 9, 10, 11, 12, 13 or 14 days. In some instances, a fixed dose of ZP1848 may be used in accordance with a dosing pattern disclosed herein, i.e., a dose which is the same regardless of the body weight of the patient, given once or twice weekly. By way of example, the fixed dose may be a dose of 5 mg, 6 mg, 7 mg, 8 mg, 9, mg, 10 mg, 11 mg, 12 mg, 13 mg, 14 mg, or 15 mg. Conveniently a fixed dose of 10 mg may be used. The use of fixed dosing has the advantage of increasing compliance and reducing the risk of patient dosing errors, including risks of miscalculating a weight-based dose to be administered.

In preferred embodiments, the formulation is a ready-to-use formulation as described in WO 2020/065064. The term “ready-to-use” as used herein refers to a formulation that does not require constitution or dilution with a prescribed amount of diluent, e.g., water for injection or other suitable diluent, before use by the designated route of administration.

As described herein, the liquid formulations of the GLP-2 analogues of the present invention include a buffer, a non-ionic tonicity modifier and arginine q.s. to provide the pH of the final formulation. In accordance with normal pharmaceutical practice, the formulations of the present invention are sterile and/or free from reducing agent. In some cases, the liquid formulations of the present invention are aqueous, liquid formulations. In some cases, the liquid formulations of the present invention are non-aqueous, liquid formulations.

The term “buffer” as used herein denotes a pharmaceutically acceptable excipient which stabilizes the pH of a pharmaceutical formulation. Suitable buffers are well known in the art and can be found in the literature. The screening experiments in the examples show that the formulations of the present invention preferably include a buffer selected from a histidine buffer, mesylate buffer, acetate buffer, glycine buffer, lysine buffer, TRIS buffer, Bis-Tris buffer and MOPS buffer as these buffers provided stable formulations in which the GLP-2 analogues dissolved and did not become viscous, cloudy, or precipitate the peptide drug. In preferred embodiments, the buffer is a histidine buffer, e.g., L-histidine. Generally, the buffer will be present at a concentration of about 5 mM to about 50 mM, more preferably at a concentration of about 5 mM to about 25 mM, and most preferably at a concentration of about 15 mM. Preferably the buffer is not a phosphate buffer, a citrate buffer, citrate/Tris buffer and/or succinate buffer.

The term “tonicity modifier” as used herein denotes pharmaceutically acceptable tonicity agents that are used to modulate the tonicity of the formulation. The formulations of the present invention are preferably isosmotic, that is they have an osmotic pressure that is substantially the same as human blood serum. The tonicity modifiers used in the formulations are preferably non-ionic tonicity modifiers and are preferably selected from the group consisting of mannitol, sucrose, glycerol, sorbitol, and trehalose. A preferred non-ionic tonicity modified is mannitol, e.g., D-mannitol. The concentration of the tonicity modifier will be dependent on the concentration of other components of the formulation, especially where the formulation is intended to be isosmotic. Typically, the non-ionic tonicity modifier will be employed at a concentration of about 90 mM to about 360 mM, more preferably at a concentration of about 150 mM to about 250 mM, and most preferably at a concentration of about 230 mM.

Generally, the components and amounts of the liquid formulations are chosen to provide a formulation with a pH of about 6.6 to about 7.4, more preferably a pH of about 6.8 to about 7.2, and most preferably a pH of about 7.0. Arginine may be added quantum sufficit (q.s.) to adjust pH so that it is within a desired pH range. From the experiments shown in the examples, it is preferred that the pH adjustment is not done using hydrochloric acid or sodium hydroxide.

In one embodiment, the liquid formulations consist of ZP1848, e.g. an acetate salt thereof, at a concentration of about 2 mg/mL to about 30 mg/mL a buffer selected from the group consisting of a histidine buffer, mesylate buffer, acetate buffer, glycine buffer, lysine buffer, TRIS buffer, Bis-Tris buffer and MOPS buffer, the buffer being present at a concentration of about 5 mM to about 50 mM, a non-ionic tonicity modifier selected from the group consisting of mannitol, sucrose, glycerol, sorbitol and trehalose at a concentration of about 90 mM to about 360 mM, arginine q.s. to provide a pH of about 6.6 to about 7.4.

In one embodiment, the liquid formulations consist of ZP1848, e.g. an acetate salt thereof, at a concentration of about 2 mg/mL to about 30 mg/mL, a buffer selected from the group consisting of a histidine buffer, mesylate buffer and acetate buffer, the buffer being present at a concentration of about 5 mM to about 50 mM, a non-ionic tonicity modifier selected from the group consisting of mannitol, sucrose, glycerol and sorbitol at a concentration of about 90 mM to about 360 mM, arginine q.s. to provide a pH of about 6.6 to about 7.4.

In a further embodiment, the liquid formulations comprise ZP1848, e.g., an acetate salt thereof, at a concentration of about 20 mg/mL, histidine buffer at a concentration of about 15 mM, mannitol at a concentration of about 230 mM, and arginine q.s. to provide a pH of about 7.0.

In a further embodiment, the liquid formulations comprise ZP1848, e.g., an acetate salt thereof, at a concentration of about 20 mg/mL, histidine buffer at a concentration of about 15 mM, mannitol at a concentration of about 230 mM and the pH is about 7.0.

In a further embodiment, the liquid formulations comprise ZP1848-acetate or H-HGEGTFSSELATILDALAARDFIAWLIATKITDKKKKKK-NH₂ acetate (SEQ ID NO: 6) at a concentration of about 20 mg/mL, histidine buffer at a concentration of about 15 mM, mannitol at a concentration of about 230 mM, and arginine q.s. to provide a pH of about 7.0.

In a further embodiment, the liquid formulations comprise ZP1848-acetate or H-HGEGTFSSELATILDALAARDFIAWLIATKITDKKKKKK-NH₂ acetate (SEQ ID NO: 6) at a concentration of about 20 mg/mL, histidine buffer at a concentration of about 15 mM, mannitol at a concentration of about 230 mM and the pH is about 7.0.

In a further embodiment, the liquid formulations comprise an acetate salt of a glucagon-like peptide 2 (GLP-2) analogue having the formula:

(H-HGEGTFSSELATILDALAARDFIAWLIATKITDKKKKKK-NH2),
x(CH₃COOH) where x is 1.0 to 8.0,

at a concentration of about 20 mg/mL, histidine buffer at a concentration of about 15 mM, mannitol at a concentration of about 230 mM and the pH is about 7.0.

In a further embodiment, the liquid formulations comprise an acetate salt of a glucagon-like peptide 2 (GLP-2) analogue having the formula:

(H-HGEGTFSSELATILDALAARDFIAWLIATKITDKKKKKK-NH2),
x(CH₃COOH) where x is 1.0 to 8.0.,

at a concentration of about 20 mg/mL, histidine buffer at a concentration of about 15 mM, mannitol at a concentration of about 230 mM and the pH is about 7.0, in a once or twice daily dosing regimen.

In a further embodiment, the liquid formulations comprise an acetate salt of a glucagon-like peptide 2 (GLP-2) analogue having the formula:

(H-HGEGTFSSELATILDALAARDFIAWLIATKITDKKKKKK-NH2),
x(CH₃COOH) where x is 1.0 to 8.0,

at a concentration of about 20 mg/mL, histidine buffer at a concentration of about 15 mM, mannitol at a concentration of about 230 mM and the pH is about 7.0, in a once or twice weekly dosing regimen.

In some cases, the liquid formulations of the present invention further comprise a preservative. In some cases, the preservative is one selected from the group consisting of benzalkonium chloride, chloro butanol, methyl paraben and potassium sorbate. Generally, the preservative is present in a concentration of about 0.1% to about 1% of the final formulation volume.

### Medical Conditions

The peptides of the present invention are useful as a pharmaceutical agent for treating an individual suffering from short bowel syndrome.

ZP1848 or a salt thereof may be useful therapeutically for the treatment of short bowel syndrome (SBS), also known as short gut syndrome or simply short gut, which results from surgical resection, congenital defect or disease-associated loss of absorption in the bowel in which patients are subsequently unable to maintain fluid, electrolyte, and nutrient balances on a conventional diet. Despite an adaptation that occurs generally in the two years after resection, SBS patients have reduced dietary uptake and fluid loss.

The class of human patients with SBS includes patients having SBS-intestinal failure (SBS-IF) and patients who are on the border between having SBS-intestinal insufficiency (SBS-II) and SBS-intestinal failure (SBS-IF). In some cases, patients having SBS-intestinal failure (SBS-IF) are also called SBS-PS when they are dependent on parenteral support, and the patients having SBS-intestinal insufficiency (SBS-II) are also called SBS non-PS if they are not depending on parenteral support. In the studies reported in the examples, the present inventors surprisingly found that treatment with the GLP-2 analogue according to the present invention allowed a subpopulation of treated patients to be weaned off the need for PS, i.e., treating patients who started treatment as SBS-PS and who became SBS non-PS after treatment. The data in the examples shows that treatment with ZP1848 allowed patients to become SBS non-PS for a significant period of at least 1 year, at least 2 years, or at least 3 years or more (data not shown), even if administration of the GLP-2 analogue needs to be continued after the patient becomes SBS non-PS, even indefinitely.

The spectrum of patient types with SBS is reviewed in Jeppensen, Journal of Parenteral and Enteral Nutrition, 38(1), 8S-13S, May 2014, doi: 10.1177/0148607114520994. A further division of SBS patient types can be made along the lines described in Schwartz et al., Clinical and Translational Gastroenterology (2016) 7, e142; doi:10.1038/ctg.2015.69. This separates SBS patients into early responders and late/slow responders. It is presently believed that the early responders are the ones who exhibit an early effect on treatment with a GLP-2 analogue such as ZP1848 caused by, among other effects, an increase in the width/diameter of the small intestine, while the late or slow responders are the patients which mostly or first benefit to the treatment with a GLP-2 analogue caused by an increase in the length of the small intestine. The determination of whether a patient is an early or a late responder may be used to determine the duration of the treatment regime with the GLP-2 analogue, the timing of any clinical decision to reduce parenteral support and the interval between testing to determine whether a reduction in parenteral support is possible. Accordingly, in one embodiment, the patient is a late or slow responder. The length of the small intestines may for example be measured by CT scan (computed tomography scan), MRI (magnetic resonance imaging), histology, laparoscopic or other measurements or techniques known in the art.

GLP-2 analogue treatment can result in increasing the intestinal mass or longitudinal growth of the intestines in a patient, e.g., in a human patient, especially the small intestine. ZP1848 or a salt thereof is capable of increasing the longitudinal growth of the intestines relative to a control treatment, as shown in WO 2018/229252.

This capability is of particular value in patients with SBS as this will lead to increased absorptive capacity also after treatment is stopped. Such patient would be treated for at least 1 to 3 years, such as at least 1 to 4 years, such as 1 to 10 years, such as 1 to 20 years, such as 1 to 35 years with the objective of inducing longitudinal growth of the intestines.

As already described herein, SBS patients who are on the border between intestinal insufficiency (SBS-II) or SBS-PS patients and intestinal failure (SBS-IF) or SBS non-PS may therefore have particular value from having their intestines lengthened over a 1 to 3 year treatment course, whereafter their risk if intestinal failure is decreased, for example involving weekly or twice weekly dosing over the period of treatment. This involves less risk for central catheter needs and the risk of sepsis associated with its use.

The GLP-2 analogue may also be used for the treatment of malnutrition, for example resulting from cachexia and anorexia.

As set out in the examples, QoL may be assessment using a Patient Global Impresson of Change (PGIC) scale (see https://www.fda.gov/media/116277/download and https://www.fda.gov/media/116281/download). The PGIC evaluates overall health status as perceived by the patient in a seven-point single-item scale ranging from ‘very much worse’ to ‘very much improved’, i.e., yes/no meaning that the patients are responders if they have ticked either “very much improved” or “much improved” in their PGIC questionnaire. The PGIC scale used in the EASE-SBS trial program was a 7-point Likert scale with patients asked to tick one box in response to the question:

Since the start of the trial, my overall status is:
(1) Very Much Improved
(2) Much Improved
(3) Minimally Improved
(4) No Change
(5) Minimally Worse
(6) Much Worse
(7) Very Much Worse

PGIC improvement was defined as responding “much improved” or “very much improved” on the 7-point Likert Scale for each of the weeks 4, 12, 20, and 24. Improvement between each glepaglutide treatment regimen compared to placebo was tested using the CMH test adjusting for the randomization stratification factor.

As shown in the Examples, the patient subpopulation of the present invention who are defined as being SBS non-PS may additionally be characterised by a PGIC for example when PGIC was assessed at week 24. This meant that of the subpopulation of patients weaned off of PS, 8/9 of them reported a PGIC status that was “much improved” or “very much improved”.

### EXAMPLES

The following examples are provided to illustrate preferred aspects of the invention and are not intended to limit the scope of the invention. The GLP-2 analogues administered according to the dosage regimes described herein can be made according to the methods such as solid phase peptide synthesis described in WO 2006/117565, the content of which is expressly incorporated by reference in its entirety.

### Example 1: Use of Glepaglutide in the Treatment of SBS, EASE SBS 1 Clinical Trial (EASE-1)

### Introduction

Glepaglutide (ZP1848, glepaglutide₁₋₃₉) is a potent long acting GLP-2 analogue for use in the treatment of short bowel syndrome (SBS). Glepaglutide comprises 39 L-amino acids, all of which are naturally occurring. Glepaglutide has 9 amino acid substitutions compared to native GLP-2 and a C-terminal tail consisting of 6 lysine residues, all which enables a stable long-lasting liquid formulation. Following subcutaneous injection of glepaglutide, two functionally active metabolites i.e., ZP2469 (1848₁₋₃₄) and ZP2711 (ZP1848₁₋₃₅) are formed from cleavages within the C-terminus.

### Methods

This phase 3 trial was a pivotal, multicenter, placebo controlled, randomized, parallel-group, double-blind and fixed dose, designed to confirm the efficacy of glepaglutide in reducing the parenteral support (PS) volume in SBS patients and to evaluate the efficacy of glepaglutide on other efficacy endpoints as well as the safety and tolerability of glepaglutide in patients with SBS. A total of 106 SBS patients were evenly randomized to 24 weeks of treatment with glepaglutide 10 mg once weekly, twice weekly or placebo. The clinical trial is registered at clinicaltrials.gov: NCT03690206 (EASE SBS 1 trial).

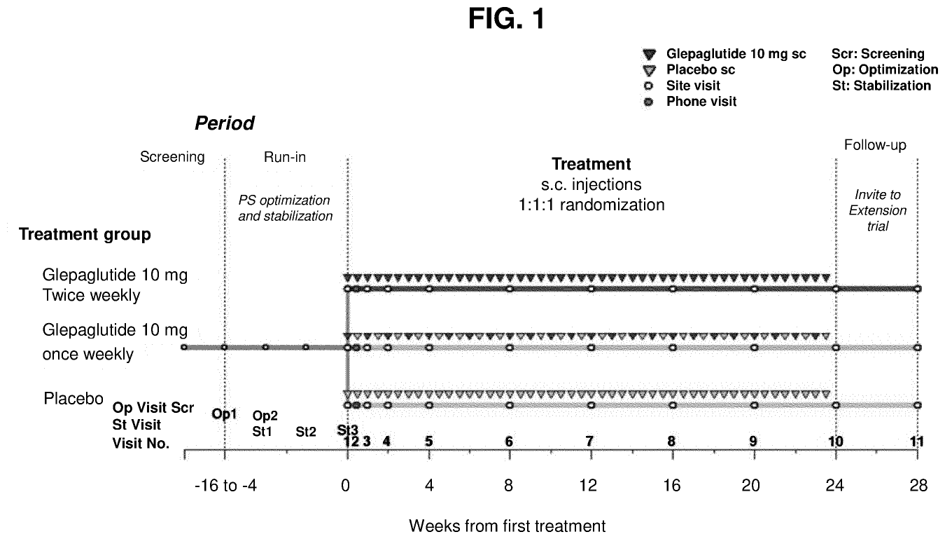

As shown in FIG. 1, the trial was designed as a 3-arm treatment, parallel-group design with a 1:1:1 randomization scheme (2 active treatment groups [once and twice weekly] and placebo) was chosen to compare the dosing regimen.

### Patient Population

Key patient inclusion criteria included:
Diagnosis of SBS defined as remaining small bowel in continuity of estimated less than 200 cm [equal to 79 inches] with the latest intestinal resection being at least 6 months prior to screening and where the patient is considered stable with regard to PS needs. No restorative surgery planned in the trial period;
PS requirement of at least 3 days per week as assessed prior to Screening and at the end of the Optimization and Stabilization Phases;
Willing to adhere to an individual pre-defined drinking menu and urine measurement during the 48-hour measuring intervals;
Age≥18 years and ≤90 years at Screening; and
At randomization: Maintains a stable PS volume for at least 2 weeks prior to randomization.

### Parenteral Support (PS) Optimization and Stabilization Phase

For up to 4 weeks prior to randomization and parenteral support baseline measurements, investigators optimized the parenteral support volume of all patients. Optimization was followed by a 2-week to 4-week (±4 days) stabilization phase.

A patient was considered stable if all of the following criteria were met: a) actual PS usage (volume and content) matches prescribed PS (±10% deviation in volume is acceptable); and b) 48-hour urine volumes at 2 consecutive visits within a 2-week interval (±4 days, i.e., visits should be 10 to 18 days apart) were similar (a maximum of ±25% deviation is acceptable), while the oral fluid intake was constant (the two 48-hour oral intakes differ less than 10%) and maximum 3.5 L per day and c) urine volume is on average≥1 L and ≤2.5 L per day.

An individual drinking menu was defined by the patient and the Investigator during the Screening period and until the end of the Optimization Phase. All patients were equipped with an electronic diary (eDiary) for recording of trial relevant data/information.

### Baseline PS Volume

Unless otherwise specified, baseline is defined as Day 1, prior to first dosing of trial product.

The baseline PS volume (L/week) is defined as the actual PS volume received during the 7-day period prior to Visit 1 (Day 1). The baseline daily urine volume (L per day) is defined as the average of the last two 48-hour urine volume measures from the Stabilization Phase.

### Main Trial Period

Visit 1 was done within 2 weeks after the last Stabilization Phase visit. All eligible patients who completed the Optimization and Stabilization Phases were randomized in a 1:1:1 manner to receive either: a) glepaglutide 10 mg twice weekly, b) glepaglutide 10 mg once weekly and placebo once weekly, or c) placebo SC twice weekly for the following 24 weeks.

During the 24-week Treatment Phase, PS need was evaluated by 48-hour balance periods involving urine measurements and during which patients were required to keep to an individually pre-defined drinking menu (timing, volume, and content) and documented this in the eDiary.

The actual volume of PS was recorded on an ongoing basis in electronic diaries (eDiaries) by the patients. The Investigator recorded the type, content, and volume of the PS being used in the eCRF. Once trial drug treatment was initiated, PS volume could be adjusted at trial visits (at Weeks 1, 2, 4, 8, 12, 16, 20, and 24) if the criteria for adjustment were met and according to a predefined algorithm for PS volume reduction:
IF: daily average urine volume of the current visit is at least 10% higher than baseline urine volume.
THEN: New PS volume (weekly)=Current PS volume (weekly)−7× absolute increase in daily urine volume from baseline.

The current PS volume is defined as the latest prescribed PS volume, i.e., the PS volume that the patient should be following prior to the visit.

### Trial Products, Dosage and Mode of Administration

Glepaglutide was provided in single-use vials containing 1 mL (an extractable volume of 0.5 mL) of a clear, essentially colorless solution for injection, containing 20 mg/mL glepaglutide. Patients randomized to active treatment got 10 mg (0.5 mL) glepaglutide injected either a) twice weekly or b) once weekly and placebo once weekly. Reference therapy: Placebo was provided in single-use vials containing 1 mL (an extractable volume of 0.5 mL) of clear, essentially colorless solution for injection. Patients randomized to placebo treatment got injected (0.5 mL) placebo solution twice weekly.

### Intervention Groups and Duration

Duration of treatment was 24 weeks across treatment groups:
a) Glepaglutide twice weekly: 10 mg glepaglutide on Day 1 and on Day 4 or 5 (same days of the week every week)
b) Glepaglutide once weekly: 10 mg glepaglutide or placebo on Days 1 and on Day 4 or 5 (same days of the week every week)
c) Placebo: placebo on Day 1 and on Day 4 or 5 (same days of the week every week)

The clinical study protocol, its amendments/updates and the informed consent forms (ICFs) and their amendments were reviewed and approved by the Health Authorities (HAs) and the Independent Ethics Committees (IECs) or Independent Review Boards (IRBs) of the countries where the trial was run prior to the screening.

### Patient Global Impresson of Change (PGIC)—Patient-Reported Outcomes (PROs)

The PGIC was used to investigate the effects of treatment on health-related quality of life (HRQoL).

Questionnaires were completed in paper format at site visits prior to any other trial related assessment. The PROs were completed by patients enrolled in the trial without assistance of site personnel. The PROs were completed at home before the patient attended the visit to the clinic. Patients were instructed to complete the PRO in a private area without influence from trial team members or accompanied by family or friends. No one was allowed to answer or interpret items for the patient. The Investigator or a delegated trial team member was allowed to read items/answers options to the patient aloud if the patient was unable to read. The Investigator or delegated trial team member instructed the patient to complete every item in the PROs and explained that there are no right or wrong answers. The Investigator or a delegated trial team member instructed the patient to give the best answer they can and explained that all individual responses were to remain confidential.

Immediately after completion, the PROs were reviewed by the Investigator (or designee) for completeness and potential adverse events (AEs). When reviewing the PROs for AEs, the Investigator was instructed not to influence nor question the patient on the content of their response to PRO questions. The review of the PROs was documented. If entries were missing in the PROs, the patient was asked to answer all questions, with care taken not to bias the patient.

The Investigator and/or delegated trial team members received training and instruction in completion of the PROs prior to the conduct of the trial.

Patients reported in the eDiary their number of bowel movements/stoma bag emptying during the 48-hour balance periods.

### Improvement at Weeks 4, 12, 20, and 24

The PGIC scale used in the EASE-SBS trial program was a 7-point Likert scale with patients asked to tick one box in response to the question:

“Since the start of the trial, my overall status is:
(1) Very Much Improved
(2) Much Improved
(3) Minimally Improved
(4) No Change
(5) Minimally Worse
(6) Much Worse
(7) Very Much Worse”

PGIC improvement was defined as responding “much improved” or “very much improved” on the 7-point Likert Scale for each of the weeks 4, 12, 20, and 24. Improvement between each glepaglutide treatment regimen compared to placebo was tested using the CMH test with stratification on the randomization stratification factor.

### Results

106 SBS patients were randomly assigned and treated with glepaglutide according to the protocol.

A total of 106 SBS patients with intestinal failure who were dependent on parenteral support (PS) for at least three days per week were evenly randomized to receive treatment with 10 mg glepaglutide administered either once or twice weekly, or placebo. The primary endpoint in the trial was the absolute change in weekly parenteral support volume from baseline at 24 weeks.

Glepaglutide given twice weekly significantly reduced the total weekly volume of parenteral support at 24 weeks as compared to placebo (p=0.0039). When administered once weekly, glepaglutide treatment also resulted in a numeric reduction in weekly parenteral support, however this did not achieve statistical significance. At 24 weeks, the average reduction in parenteral support from baseline was 5.13 Liters/week for patients treated with glepaglutide twice weekly and was 3.76 Liters/week for patients treated with glepaglutide once weekly. Placebo treatment resulted in a reduction in parenteral support of 2.85 Liters/week.

Clinical response, defined as a patient achieving at least 20% reduction in weekly parenteral support volume from baseline at both 20 and 24 weeks, was significantly higher with twice weekly glepaglutide compared to placebo (p=0.0243). Among patients receiving glepaglutide twice weekly 65.7% achieved a clinical response. While 45.7% and 38.9% of patients achieved a clinical response in the once weekly and placebo treatment groups, respectively.

In the twice weekly dosing group, 14% of patients (n=5) were completely weaned off parenteral support (enteral autonomy). In total 9 patients treated with glepaglutide achieved enteral autonomy, while no placebo treated patients were able to discontinue parenteral support.

### Clinical Classification of Patients Based on PS Volume (ESPEN Guidelines)

On the basis of the energy and volume of the required PS, the patients are categorized into 16 combinations (adapted from Pironi L, Arends J, Bozzetti F, et al. ESPEN guidelines on chronic intestinal failure in adults. Clin Nutr 2016; 35(2): 247-307; see Table 1 below).

TABLE 1
Clinical classification of chronic Intestinal failure
and the volume of the required intravenous supplementation,
IF is categorized into 16 combinations

| IV energy supplementation^{b} | Volume of IV supplementation^{a} (mL) | | | |
|---|---|---|---|---|
| (kcal/Kg body weight) | ≤1000 | 1001-2000 | 2001-3000 | >3000 |
| 0 (A) | A1 | A2 | A3 | A4 |
| 1-10 (B) | B1 | B2 | B3 | B4 |
| 11-20 (C) | C1 | C2 | C3 | C4 |
| >20 (D) | D1 | D2 | D3 | D4 |

^{a}Calculated as daily mean of the total volume infused per week ¼ (volume per day of infusion × number of infusions per week)/7.
^{b}Calculated as daily mean the total energy infused per week ¼ (energy per day of infusion × number of infusions per week)/7/Kg.

### Anatomical SBS CLASS

The patients were also group according to their remaining bowel sections
Group 1: end-jejunostomy or ileostomy
Group 2: jejuno-colic anastomosis
Group 3: jejuno-ileo-colic anastomosis

As shown in Table 2 below, glepaglutide given once (OW) or twice (TW) weekly completely weaned off parenteral support (enteral autonomy) after 24 weeks of treatment. In total 9 patients treated with glepaglutide achieved enteral autonomy, while no placebo treated patients were able to discontinue parenteral support (data not shown).

TABLE 2

| | | | | | Parenteral | |
|---|---|---|---|---|---|---|
| | | | | Parenteral | support | |
| Patient | | Anatomical | ESPEN's | support | after 24 | PGIC at |
| number | Treatment | SBS CLASS | guidelines | baseline | week | Week 24 |
| 1 | GLEPA-TW | Group 2 | A1 | 3 | 0 | Very much |
| | | | | | | improved |
| 2 | GLEPA-TW | Group 2 | C1 | 4.5 | 0 | No |
| | | | | | | change |
| 3 | GLEPA-TW | Group 2 | C2 | 7.5 | 0 | Much |
| | | | | | | improved |
| 4 | GLEPA-TW | Group 2 | B2 | 6.4 | 0 | Much |
| | | | | | | improved |
| 5 | GLEPA-TW | Group 1 | B3 | 3 | 0 | Much |
| | | | | | | improved |
| 6 | GLEPA-OW | Group 2 | C3 | 14 | 0 | Much |
| | | | | | | improved |
| 7 | GLEPA-OW | Group 1 | D1 | 6 | 0 | Much |
| | | | | | | improved |
| 8 | GLEPA-OW | Group 3 | D2 | 8.75 | 0 | Much |
| | | | | | | improved |
| 9 | GLEPA-OW | Group 2 | B2 | 8.4 | 0 | Much |
| | | | | | | improved |

### Example 2: Extension Trial Assessing the Longi-Term Safety and Efficacy of Glepaglutide in Patients with Short Bowel Syndrome (SBS), EASE SBS 2 (EASE-2)

Following the EASE-1 trial, a Double-blind, Phase 3, Extension Trial Assessing the Long-term Safety and Efficacy of Glepaglutide in Patients with Short Bowel Syndrome (SBS)

### Objectives

The primary objective of this trial was to evaluate the long-term safety of glepaglutide treatment in short bowel syndrome (SBS) patients.

The secondary objectives of this trial were:
To evaluate the long-term efficacy of glepaglutide treatment in SBS patients
To evaluate the long-term immunogenicity of glepaglutide and its impact on pharmacokinetics (PK), efficacy, and safety in SBS patients
To evaluate quality of life for SBS patients treated with glepaglutide.

### Trial Design

This was a long-term, double-blind, extension trial in patients who completed the full 24-week treatment phase of the lead-in trial (‘EASE-1’) described above in Example 1 and who consented to participate in this extension trial called ‘EASE-2’. Furthermore, it was allowed that patients were screened directly into EASE-2 to obtain additional long-term safety and efficacy data.

Duration of treatment in this extension trial was 2 years. All patients received glepaglutide treatment at a dose regimen of 10 mg twice weekly (TW) or once weekly (OW). Patients receiving TW or OW glepaglutide in EASE-1 continued their blinded treatment. Patients receiving placebo in EASE-1 were re-randomized in a 1:1 ratio to blinded treatment with 10 mg glepaglutide TW or OW, and patients screened directly into EASE-2 were randomized 1:1 to blinded treatment with 10 mg glepaglutide TW or OW. Both treatment arms involved TW dosing (glepaglutide or glepaglutide+placebo) to maintain the blinding.
Glepaglutide TW: 10 mg glepaglutide on Day 1 and on Day 4 or Day 5 (same days of the week every week).
Glepaglutide OW: 10 mg glepaglutide or placebo on Day 1 and on Day 4 or Day 5 (same days of the week every week).

Patients screened directly into EASE-2 came to a screening visit and followed a run-in phase identical to that in EASE-1 before randomization. This was to ensure a reliable baseline for assessing the efficacy of glepaglutide treatment in reducing parenteral support (PS) requirements.

During the trial, the actual volume and type of PS were recorded in the electronic diary (eDiary) on an ongoing basis by the patients. The investigator recorded the type, content, and volume of the prescribed PS.

PS volume could be adjusted at or in-between visits according to the below algorithm, which was identical to the one used for EASE-1:
IF: daily urine volume of the current visit is at least 10% higher than baseline urine volume
THEN: New PS volume (weekly)=Current PS volume (weekly)−(7× absolute change in daily urine volume from baseline).

For patients rolling over from EASE-1, the baseline urine volume (L per day) for EASE-2 was defined as the average of the last two 48-hour urine volume measures from the stabilization phase in EASE-1. For patients screened directly into EASE-2, the baseline urine volume was defined as the average of the two 48-hour urine volumes prior to visit 1 in EASE-2. During the 24-month treatment phase, patients recorded the urine volume in the eDiary during 48-hour measurement periods prior to trial visits while adhering to an individually pre-defined drinking menu.

From visit 7 onwards (i.e., after 12 weeks of investigational product treatment), patients recruited at selected sites of EASE-2 were to be included (after separate consent) in a voluntary PK sub-study with more extensive pharmacokinetics (PK) sampling to provide additional information about the PK profile of glepaglutide and its primary active metabolites in SBS patients. The aim was to include approximately 45 patients. At the time of the interim cut-off, 12 patients (6 in each treatment group) participated in the PK sub-study.

After completing the treatment phase, all patients were offered to enter an additional extension trial called ‘EASE-3’ and continue to receive glepaglutide. For patients not entering EASE-3, a follow-up visit was conducted 4 weeks after completion of the treatment phase in EASE-2.

In addition to the 9 patients who weaned off at Week 24 as described in Example 1, four patients who were initially randomised to placebo (i.e. glepaglutide naïve patients), weaned off after 6 months active treatment in the Extension trial EASE 2. Relevant baseline characteristics for these patients are shown in Table 3 below.

TABLE 3

| | | | | | Parenteral |
|---|---|---|---|---|---|
| | Treatment | Anatomical | | Parenteral | support |
| Patient | in extension | SBS | ESPEN's | support | after 24 |
| number | trial | CLASS | guidelines | baseline | week |
| 10 | GLEPA-OW | Group 2 | D4 | 5.25 | 0 |
| 11 | GLEPA-OW | Group 2 | B1 | 4.8 | 0 |
| 12 | GLEPA-OW | Group 2 | D3 | 10.2 | 0 |
| 13 | GLEPA-OW | Group 2 | C2 | 7.8 | 0 |

While the invention has been described in conjunction with the exemplary embodiments described above, many equivalent modifications and variations will be apparent to those skilled in the art when given this disclosure. Accordingly, the exemplary embodiments of the invention set forth are considered to be illustrative and not limiting. Various changes to the described embodiments may be made without departing from the spirit and scope of the invention. All documents cited herein are expressly incorporated by reference.

1. A glucagon-like peptide 2 (GLP-2) analogue for use in a method for the treatment of a human patient suffering from short bowel syndrome (SBS) and receiving parenteral support (PS), wherein the GLP-2 analogue is represented by the formula:
(SEQ ID NO: 1)
H-HGEGTFSSELATILDALAARDFIAWLIATKITDKKKKKK-NH₂,
(1848)
or a pharmaceutically acceptable salt thereof, the method comprising:
(a) providing a human patient suffering from short bowel syndrome and receiving parenteral support (PS);
(b) for a period of time administering by subcutaneous injection once or twice weekly 10 mg of a GLP-2 analogue into the patient, while the patient continues to receive the PS; and
(c) following step (b), weaning the patient off the PS.

2. The glucagon-like peptide 2 (GLP-2) analogue for use in a method for the treatment according to claim 1, wherein the period of time in step (b) is between 20 weeks and 52 weeks.

3. The glucagon-like peptide 2 (GLP-2) analogue for use in a method for the treatment according to claim 2, wherein the period of time in step (b) is between 24 weeks and 36 weeks.

4. The glucagon-like peptide 2 (GLP-2) analogue for use in a method for the treatment according to any one of claims 1 to 3, further comprising:
(d) following step (c) administering by subcutaneous injection once or twice weekly 10 mg of the GLP-2 analogue into the patient while the patient receives no PS.

5. The glucagon-like peptide 2 (GLP-2) analogue for use in a method for the treatment according to any one of claims 1 to 4, wherein the patient is treated once or twice weekly for at least 1 to 3 years.

6. The glucagon-like peptide 2 (GLP-2) analogue for use in a method for the treatment according to any one of claims 1 to 5, wherein the patient retains SBS non-PS status for at least 1 year, optionally for at least 2 years and optionally for at least 3 years or more.

7. The glucagon-like peptide 2 (GLP-2) analogue for use in a method for the treatment according to any one of claims 1 to 6, wherein the improvement in quality of life (QoL) of the patient is assessed using a Patient Global Impression of Change (PGIC) status.

8. The glucagon-like peptide 2 (GLP-2) analogue for use in a method for the treatment according to claim 7, wherein the PGIC status uses a 7-point Likert Scale with responding patients reporting a much improved or very much improved status as compared to treatment with placebo.

9. The glucagon-like peptide 2 (GLP-2) analogue for use in a method for the treatment according to claim 7 or claim 8, wherein the improved PGIC status of the patient is observable at week 24 of the treatment.

10. The glucagon-like peptide 2 (GLP-2) analogue for use in a method for the treatment according to any one of claims 1 to 9, wherein step (b) comprises administering by subcutaneous injection once weekly 10 mg of the GLP-2 analogue into the patient.

11. The glucagon-like peptide 2 (GLP-2) analogue for use in a method for the treatment according any one of claims 1 to 10, wherein step (b) comprises administering by subcutaneous injection twice weekly 10 mg of the GLP-2 analogue into the patient.

12. The glucagon-like peptide 2 (GLP-2) analogue for use in a method for the treatment according to any one of claims 1 to 11, wherein the patient has undergone an end-jejunostomy or ileostomy.

13. The glucagon-like peptide 2 (GLP-2) analogue for use in a method for the treatment according to any one of claims 1 to 12, wherein the patient has undergone a jejuno-colic anastomosis.

14. The glucagon-like peptide 2 (GLP-2) analogue for use in a method for the treatment according to any one of claims 1 to 12, wherein the patient has undergone a jejuno-ileo-colic anastomosis.

15. The glucagon-like peptide 2 (GLP-2) analogue for use in a method for the treatment according to any one of claims 1 to 14, wherein step (b) comprises the patient receiving PS at an ESPEN guideline level of any one of A1, B1, C1, D1, A2, B2, C2, D2, A3, B3, C3, D3, A4, B4, C4, or D4.

16. A method of treating short bowel syndrome in a patient in need thereof said method comprising:
(a) providing a human patient suffering from short bowel syndrome and receiving parenteral support (PS);
(b) for a period of time administering by subcutaneous injection once or twice weekly 10 mg of a GLP-2 analogue into the patient, wherein the GLP-2 analogue is represented by the formula:
(SEQ ID NO: 1)
H-HGEGTFSSELATILDALAARDFIAWLIATKITDKKKKKK-NH₂,
(1848)
or a pharmaceutically acceptable salt thereof while the patient continues to receive the PS; and
(c) following step (b), weaning the patient off the PS.

17. The method according to claim 16, wherein the period of time in step (b) is between 20 weeks and 52 weeks.

18. The method according to claim 17, wherein the period of time in step (b) is between 24 weeks and 36 weeks.

19. The method according to any one of claims 16 to 18, further comprising:
(d) following step (c) administering by subcutaneous injection once or twice weekly 10 mg of the GLP-2 analogue into the patient while the patient receives no PS.

20. The method according to any one of claims 16 to 19, wherein the patient is treated once or twice weekly for at least 1 to 3 years.

21. The method according to any one of claims 16 to 20, wherein the patient retains SBS non-PS status for at least 1 year, optionally for at least 2 years and optionally for at least 3 years or more.

22. The method according to any one of claims 16 to 21, wherein the improvement in quality of life (QoL) of the patient is assessed using a Patient Global Impression of Change (PGIC) status.

23. The method according to any one of claims 16 to 22, wherein the PGIC status uses a 7-point Likert Scale with responding patients reporting a much improved or very much improved status as compared to treatment with placebo.

24. The method according to claim 22 or claim 23, wherein the improved PGIC status of the patient is observable at week 24 of the treatment.

25. The method according to any one of claims 16 to 24, wherein step (b) comprises administering by subcutaneous injection once weekly 10 mg of the GLP-2 analogue into the patient.

26. The method according to any one of claims 16 to 25, wherein step (b) comprises administering by subcutaneous injection twice weekly 10 mg of the GLP-2 analogue into the patient.

27. The method according to any one of claims 16 to 26, wherein the patient has undergone an end-jejunostomy or ileostomy.

28. The method according to any one of claims 16 to 27, wherein the patient has undergone a jejuno-colic anastomosis.

29. The method according to any one of claims 16 to 28, wherein the patient has undergone a jejuno-ileo-colic anastomosis.

30. The method according to any one of claims 16 to 29, wherein step (b) comprises the patient receiving PS at an ESPEN guideline level of any one of A1, B1, C1, D1, A2, B2, C2, D2, A3, B3, C3, D3, A4, B4, C4, or D4.


SEQUENCE LISTING (6 sequences)

SEQ ID NO: 1 - protein, 39 aa, synthetic construct
HGEGTFSSELATILDALAARDFIAWLIATKITDKKKKKK

SEQ ID NO: 2 - protein, 33 aa, synthetic construct
HADGSFSDEMNTILDNLAARDFINWLIQTKITD

SEQ ID NO: 3 - protein, 34 aa, synthetic construct
HGEGTFSSELATILDALAARDFIAWLIATKITDK

SEQ ID NO: 4 - protein, 35 aa, synthetic construct
HGEGTFSSELATILDALAARDFIAWLIATKITDKK

SEQ ID NO: 5 - protein, 33 aa, synthetic construct
HGDGSFSDEMNTILDNLAARDFINWLIQTKITD

SEQ ID NO: 6 - protein, 39 aa, synthetic construct; identical to SEQ ID NO: 1